(19) United States
(12) Patent Application Publication
(10) Pub. No.: US 2021/0077783 A1
(43) Pub. Date: Mar. 18, 2021

(54) INTRAVENOUS ACCESSORY DEVICE AND METHOD OF USING SAME

(71) Applicant: TECHNION RESEARCH & DEVELOPMENT FOUNDATION LIMITED, Haifa (IL)

(72) Inventors: Ofer BINAH, Nofit (IL); Uri BINAH, Nofit (IL)

(21) Appl. No.: 16/970,492

(22) PCT Filed: Feb. 19, 2019
(86) PCT No.: PCT/IL2019/050197

§ 371 Date: Aug. 17, 2020

Related U.S. Application Data
(60) Provisional application No. 62/634,835, filed on Feb. 24, 2018.

(51) Int. Cl. A61M 25/02; A61M 25/06

(52) U.S. Cl. CPC A61M 25/02; A61M 25/0606; A61M 2025/0273; A61M 2025/0246; A61M 2207/00

(57) ABSTRACT

A layered patch for inserting and fixating an intravenous (IV) device, the patch including a first layer configured to adhere to a patient's skin, the first layer having a slot configured to expose a blood vessel of the patient, wherein the slot is sized and shaped to allow essentially unhindered access of the IV device to the exposed blood vessel, and wherein lateral edges of the slot include a bulged structure configured to prevent/inhibit lateral movement of the exposed blood vessel; and a second layer configured to be placed over and/or adhere to the first layer, wherein the first and second layers are attached one to another at a first end thereof.

### FIELD OF THE INVENTION

The present invention relates to intravenous accessory devices, particularly to patches for inserting and fixating an intravenous (IV) device.

### BACKGROUND OF THE INVENTION

The procedures of venous blood sampling and intravenous administration of drugs and fluids are performed on millions of patients, neonates-to-elderly, daily throughout the world. These procedures, some of which are life-saving, are performed in a variety of environmental settings, such as hospitals, battle-fields, ambulances, helicopters, car-accident sites, and at home. These procedures may be performed by medical and/or health care professionals, such as a nurse and/or a doctor. Some of the medical professionals may be better trained than others, and often operate under stressful conditions. In addition, self-intravenous administration of drugs is often required of patients. These patients have difficulties introducing the intravenous needle with one hand into a vein. Intra-blood vessel administration requires the location of a blood vessel, typically a vein, within the body of the treated subject. Once the vein is located, it must be stabilized before and during penetration by the needle. If the vein is not stabilized, it is likely to move or slide during the procedure, which can lead to failing to penetrate (or access) the lumen of the blood vessel. Such failure often leads to additional undesired puncturing of the blood vessel walls leading to hemorrhaging, pain and loss of valuable time, and patients' anxiety.

Currently, intra-blood vessel administration typically requires applying tension on the skin by stretching it with the care giver's hand in order to stabilize the blood vessel. This requires a free hand, which is often not available, particularly in cases of self-administration.

Furthermore, even if the insertion into the blood vessel was successful, securing the intravenous apparatus (such as a Venflon) to the skin, in order to avoid unintentional pulling of the needle/tube, is time-consuming and complex.

It is therefore desirable to have an apparatus aiding and/or simplifying intravenous needle insertion into a blood vessel, and securing the apparatus onto the skin.

### SUMMARY OF THE INVENTION

There is provided, in accordance with some embodiments of the present invention, a multi-purpose device configured for aiding and/or simplifying insertion of a medical device into a blood vessel of a patient as well as for securing the apparatus to the skin surface once the insertion is completed. The device includes a layered patch including a first layer configured to contact a patient's skin. The first layer includes a slot, essentially devoid of material, and configured to expose a target area on the subject skin. The slot is sized and shaped to receive an insertion device (e.g., an infusion needle) and/or to allow essentially unhindered access of an insertion device to the target area on the patient's skin. The slot in the first layer may be regarded as a “runway-shaped” window surrounding the vein and thus making the IV insertion process more visualized for the medical caregiver or for the user in cases of self-administration. The first layer has a first surface at least partially coated with an adhesive configured to removably adhere to the patient's skin in an area surrounding the target area, and a second surface opposite the first surface configured to removably adhere to a second layer of the patch and/or to a non-inserted portion of the insertion device, thus securing and/or immobilizing the insertion device after its insertion.

In accordance with some embodiments of the present invention, a width of the slot is greater than a width of the blood vessel. According to some embodiments, the slot is sized and shaped to expose a blood vessel, when adhered to a patient's skin. According to some embodiments, the width of the slot is in the range of 0.5 cm to 1.5 cm. Advantageously, the relatively large width of the slot, as opposed, for example, to slits, enables access to the target blood vessel without requiring an enlarging of the size of the open area and/or without pushing aside interfering material. In addition, the relatively large size of the slot enables the blood vessel to protrude/bulk out in the tunnel formed by the slot.

In accordance with some embodiments of the present invention, the first layer includes one or more stabilizing bulk/edge, extending from, and essentially perpendicular to, the first surface of the first layer. According to some embodiments, the one or more stabilizing bulks may extend along a length (optionally the entire length) of the slot shore/lateral edge. According to some embodiments, the one or more stabilizing bulks may form a tunnel-like structure. Advantageously, the tunnel-like structure enables receiving and/or “capturing” of a blood vessel, exposed by the slot. As a further advantage, the tunnel-like structure may prevent, inhibit, diminish, and/or reduce lateral movement of the blood vessel captured therein, thus enabling penetration of the blood vessel, by the insertion device, while ensuring that a “slipping aside” of the blood vessel, the most common cause of insertion failure, is prevented or at least significantly minimized.

According to some embodiments, the one or more stabilizing bulks may be one or more flaps, e.g., a pair of flaps on each shore of the slot.

According to some embodiments, the slot may be tapered, such that a width at the distal (open) end of the slot is larger than the width of the slot at its proximal (closed) end. Such structure may advantageously serve as a guide to the insertion of the insertion device.

According to some embodiments, the bulk may be tapered such that the height of the bulk is larger at a distal end thereof (i.e., at the end closest to the distal, open end of the slot), than the height at its proximal end. According to some embodiments, the bulk may be tapered such that the height of the bulk is smaller at a distal end thereof (i.e., at the end closest to the distal, open end of the slot), than the height at its proximal end.

According to some embodiments, the slot may be open ended at its distal end. According to some embodiments, the slot may be closed at its proximal end. According to some embodiments, a proximal end of the slot is coextensive with a first edge of the first layer. According to some embodiments, a distal end of the slot terminates proximally to the opposite edge of the first layer, such that the two halves of the first layer, divided by the slot, are interconnected at one end thereof.

According to some embodiments, the second layer may have a same size and shape than that of the first layer. Alternatively, according to some embodiments, the second layer may be larger than the first layer so that the first layer is covered by the second layer, when the second layer is placed over and/or adhered to the first layer. According to some embodiments, the second layer may include a second slot having essentially the same size and shape as the slot of the first layer. According to some embodiments, the width of the slot of the second layer may be slightly larger (e.g., up to 1.5 times larger) or slightly smaller (e.g., up to 1.5 times smaller) than the width of the slot of the first layer.

According to some embodiments, the second layer may be attached to the first layer at the edge thereof closest to the slot's proximal (closed) end.

According to some embodiments, the patch may further include a third layer configured to cover the first and second layers. According to some embodiments, the third layer may advantageously serve as a protective layer, providing a safe and clean environment. According to some embodiments, the third layer may be larger than the first and second layers. According to some embodiments, the third layer may overlay the skin, thereby further securing the adherence of the first layer to the patient's skin as well as the immobility of the insertion device.

According to some embodiments, the third layer is attached to the first and/or second layers at the edge thereof closest to the slot's proximal (closed) end.

According to some embodiments, the layers of the patch may form a single integrative patch.

According to some embodiments, the patch may include additional layers, such as, but not limited to, a fourth, a fifth or a sixth layer. Each possibility is a separate embodiment.

There is further provided, in accordance with some embodiments of the present invention, a method for inserting an insertion device (e.g., an infusion needle) into a patient, including removably adhering at least a portion of a first layer of a layered patch to the patient's skin over a blood vessel of the patient, such that the blood vessel is exposed in a slot formed in the patch's first layer. According to some embodiments, the shores/lateral edges of the slot may include bulges, which form a tunnel-like structure, configured to capture, immobilize and/or stabilize the blood vessel, when adhered to the patient's skin.

According to some embodiments, the insertion device may then be inserted and/or penetrated into the part of the patient's blood vessel exposed by the slot.

According to some embodiments, the method further includes placing a second layer of the patch over the first layer such that a non-inserted/penetrated portion of the insertion device is adhered to an upper surface of the first layer. According to some embodiments, at least a portion of the outer surface of the first layer and/or the inner surface of the second layer may include an adhesive or other attachment means configured to adhere the second layer to the first layer when juxtaposed.

According to some embodiments, the method further includes placing a third layer of the patch over the second layer of the patch such that an area of the patient's skin larger than the area covered by the first and/or second layers is covered.

According to some embodiments, the method further includes exposing and/or pulling a part of the insertion device, to which continued access is required, through a slit formed in the third layer.

According to some embodiments, there is provided herein a layered patch for inserting and fixating an intravenous (IV) device, the patch includes: a first layer configured to adhere to a patient's skin, the first layer includes a slot configured to expose a blood vessel of the patient, wherein the slot is sized and shaped to allow essentially unhindered access of the IV device to the exposed blood vessel and wherein lateral edges of the slot include a bulged structure configured to prevent/inhibit lateral movement of the exposed blood vessel; and a second layer configured to be placed over and/or adhere to the first layer, wherein the first and second layers are attached one to another at a first end thereof.

According to some embodiments, the first layer is coated with an adhesive configured to removably adhere to the patient's skin on a first, patient facing surface of the first layer.

According to some embodiments, the second layer may have the same size and shape as the first layer. According to some embodiments, the second layer may include a second slot having essentially the same size and shape as the slot of the first layer.

According to some embodiments, the patch may further include a third layer configured to be placed over and/or adhered to the first layer and/or the second layer. According to some embodiments, the third layer may be larger than the first and second layers. According to some embodiments, the third layer may include a slit through which part of the insertion device, to which repeated access is required, may emerge. According to some embodiments, the slit may be narrower than the slot, thus preventing/reducing infection at the penetration site. According to some embodiments, the third layer may be attached to the first and/or second layers at the first end thereof. According to some embodiments, the first, second and third layers are attached to one another at a first end of the patch, such that, when in use, the first end faces the proximal part of the body. According to some embodiments, the patch may include a strap configured to attach to the first end of the first, second and third layers. The strap may include a surface, upon which information may be written or otherwise noted. Such information may include the insertion date, the date in which the IV device needs to be replaced, the name of the caregiver who inserted the IV device, etc.

Furthermore, as mentioned hereinabove, even if the insertion of the needle into the blood vessel was successful, securing the IV device (such as a Venflon) to the skin, using current methods, is time consuming and complex and may lead to unintentional pulling of the needle/tube of the intravenous device. Advantageously, the three-layered structure of the patch, in accordance with some embodiments, facilitates a simple and fast yet firm securing of the IV device to the patient's skin surface by “sandwiching” the “wings” of the IV device between the first and the second patch layers and further reinforcing the attachment by applying the third layer. The simple, fast and firm securing of the IV device to the skin, as disclosed herein, in accordance with some embodiments, may be particularly advantageous, when the patient needs to self-administer a drug.

According to some embodiments, the patch is structured to self-adhere to the skin, and to expose and stabilize/immobilize/anchor the vein, without the need of a strap or other means to hold down the patch. When the vein is exposed and immobilized, the penetration of the needle becomes easier, and the risk of missing the vein is mitigated. Thus, valuable time, which is often translated to money, is saved, and the patient's pain, anxiety, discomfort and often hemorrhages, are reduced or even avoided. Moreover, in cases of unsuccessful IV insertions, a caregiver/patient will be frustrated by his/her inability to penetrate the vein, which may (psychologically) increase the likelihood of failing in a subsequent attempt. According to some embodiments, the first, second and third layers form a single integrative patch.

According to some embodiments, the first, second and/or third layers may include one or more luminescent material(s) facilitating the insertion and/or fixation of the intravenous (IV) device. The luminescent material(s) may include, for example, fluorescent material(s), phosphorescent material(s) or a combination thereof.

According to some embodiments, there is provided herein a method for inserting and fixating an intravenous (IV) device, the method includes: providing a layered patch including a first layer and a second layer; wherein the first layer includes a slot; removably adhering at least a portion of the first layer to the patient's skin, such that a target blood vessel is exposed in the slot; inserting the IV device into the blood vessel exposed by the slot; and securing the IV device to the patient's skin by placing the second layer over the first layer, such that a non-inserted portion of the IV device is immobilized between the first and second layers.

According to some embodiments, the slot may include a bulged structure on its lateral edges configured to prevent/inhibit lateral movement of the blood vessel.

According to some embodiments, the method may further include a step of placing a third layer of the patch over the second layer of the patch, such that an area of the patient's skin is larger than the area covered by the first and/or second layers.

According to some embodiments, the method may further include a step of exposing and/or pulling a part of the insertion device, to which continued access is required, through a slit in the third layer.

According to some embodiments, there is provided herein a method for producing a layered patch for inserting and fixating an intravenous (IV) device, the method includes: forming a first layer including a slot, sized and shaped to expose a blood vessel of a patient and to allow essentially unhindered access of the IV device to the exposed blood vessel, when adhered to a patient's skin; wherein lateral edges of the slot include a bulged structure configured to prevent/inhibit lateral movement of the exposed blood vessel; forming a second layer configured to be placed over and/or adhere to the first layer, attaching edges of the first and second layers at a first end thereof.

According to some embodiments, the method may further include a step of adding an adhesive on a first surface thereof, wherein the adhesive is configured to allow removably adhering the patch to the patient's skin.

According to some embodiments, the method may further include a step of forming a third layer configured to be placed over and/or adhered to the first layer and/or the second layer; wherein the third layer is larger than the first and second layers.

According to some embodiments, the method may further include a step of forming a slit in the third layer through which part of the insertion device, to which repeated access is required, may emerge, wherein the slit has a narrower width than the slot.

According to some embodiments, the method may further include a step of attaching an edge of the third layer to the edges of the first and second layers, at a first end thereof.

According to some embodiments, a width of the slot may be greater than a width of the blood vessel. According to some embodiments, a width of the slot may be smaller than a width of the blood vessel. According to some embodiments, a width of the slot may be about the same as a width of the blood vessel. According to some embodiments, the width of the slot may be in the range of 0.5 cm to 1.5 cm. According to some embodiments, the slot is closed at its proximal end and opened at its distal end.

According to some embodiments, the slot may be tapered, such that a width at the proximal end of the slot is smaller than the width of the slot at its distal end. According to some embodiments, the tapering may be stepwise.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some, or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements, or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 is a top view of a device according to an exemplary embodiment of the present invention;

FIG. 2 is a bottom view of the device illustrated in FIG. 1;

FIG. 3 is a perspective view of the device illustrated in FIG. 1 attached to a patient's arm and securing in place an inserted intravenous device;

FIG. 4 is a perspective view of the device illustrated in FIG. 1 adhered to a patient's arm prior to insertion of the intravenous device;

FIG. 5 is a perspective view of a device according to an exemplary embodiment of the present invention;

FIG. 6 is a top view of a device according to an exemplary embodiment of the present invention;

FIG. 7 is a bottom view of the device illustrated in FIG. 6;

FIG. 8 is a top view of a device according to an exemplary embodiment of the present invention;

FIG. 9 is a bottom view of the device illustrated in FIG. 8;

FIG. 10 is a top view of a device according to an exemplary embodiment of the present invention;

FIG. 11 is a bottom view of the device illustrated in FIG. 10;

FIG. 12 is a top view of a device for facilitating insertion of a medical device into a blood vessel of a patient according to an exemplary embodiment of the present invention;

FIG. 13 schematically illustrates a device for facilitating insertion of a medical device into a blood vessel of a patient with medical device flap pulled upward. according to an exemplary embodiment of the present invention;

FIG. 14 schematically illustrates a device for facilitating insertion of a medical device into a blood vessel of a patient after insertion; according to an exemplary embodiment of the present invention;

FIG. 15 schematically illustrates a device for facilitating insertion of a medical device into a blood vessel of a patient after insertion, according to an exemplary embodiment of the present invention;

FIG. 16a schematically illustrates a layered patch with a slot for inserting and fixating an intravenous (IV) device, according to an exemplary embodiment of the present invention;

FIG. 16b schematically illustrates a layered patch with a slot having a stepwise tapering for inserting and fixating an intravenous (IV) device, according to an exemplary embodiment of the present invention;

FIG. 17a is a perspective view of the device illustrated in FIG. 15 wherein a first layer thereof is attached to a patient's arm, prior to insertion of the IV device, according to an exemplary embodiment of the present invention;

FIG. 17b schematically illustrates a cross-sectional view of the device illustrated in FIG. 15 wherein a first layer thereof is attached to a patient's arm (as in FIG. 17a);

FIG. 17c is a perspective view of the device illustrated in FIG. 15 wherein the first layer thereof is attached to a patient's arm, during insertion of the IV device;

FIG. 17d is a perspective view of the device illustrated in FIG. 15 wherein the second layer thereof is adhered to the first layer, such that the IV device is secured to/immobilized on the patient's arm; and

FIG. 17e is a perspective view of the device illustrated in FIG. 15 wherein the third layer thereof is laid over the first and second layers.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a layered patch for inserting and fixating an intravenous (IV) device, the patch including a first layer configured to adhere to a patient's skin, and a second layer configured to be placed over and/or adhere to the first layer, wherein the first and second layers are attached one to another at a first end thereof. The first layer includes a slot, sized and shaped to expose a blood vessel and to allow essentially unhindered access of the IV device to the exposed blood vessel, when adhered to the patient's skin. According to some embodiments, the lateral edges of the slot may include a bulged structure configured to prevent/inhibit lateral movement of the exposed blood vessel.

As used herein the term “patch” refers to a plaster-like wrap, pad or other medical dressing configured to adhere to a subject's skin. As used herein, the term “layered patch” may refer to a patch having several layers wherein the inner most of the layers is configured to be removably adhered to a patient's skin. The layers of the patch are interconnected and/or attached to one another at one edge thereof, whereas the opposite edges of the layers are separable (like a pamphlet). According to some embodiments, the layers of the patch may be made of a same or a different material. According to some embodiments, the first and second layers may be made of woven or non-woven fabric. According to some embodiments, at least the outer surface of the third layer may be made of or include a water resistant and/or water repellant material. According to some embodiments, the first layer, the second layer and/or the third layer may be transparent or translucent to facilitate visualization of an IV device after insertion.

As used herein, the term intravenous (IV) device may refer to any device at least a portion of which is configured for penetration into a patient's blood vessel. According to some embodiments, the IV device may be an infusion needle, an intravenous cannula (e.g., Venflon), and/or a peripheral venous catheter.

As used herein, the term “slot” may refer to a cutout in the layer. According to some embodiments, the slot may be essentially rectangular, or trapeze shaped. According to some embodiments, the slot may be open ended at one end thereof, i.e. the cut may commence at the edge of the layer at one end thereof and terminate prior to reaching the opposite edge of the layer.

As used herein, the terms “bulge” and “bulged structure” may refer to any protuberance, flap or swelling protruding out from the inner surface of the first layer (facing the patient). According to some embodiments, the bulge may be formed along the lateral edges of the slot, such that a tunnel-like compartment is formed, configured to receive, hold, capture and/or immobilize the target vessel, into which penetration is desired.

According to some embodiments, the first layer is coated with an adhesive configured to removably adhere to the patient's skin on a first, patient facing surface thereof. Non-limiting examples of suitable adhesives include acrylate, including methacrylates and epoxy diacrylates (also known as vinyl resins). According to some embodiments, surfaces covered with an adhesive may be covered with a protective, removable coating prior to use.

According to some embodiments, the width of the slot is greater than a width of the blood vessel. According to some embodiments, the width of the slot is smaller than a width of the blood vessel. According to some embodiments, the width of the slot is about the same as a width of the blood vessel According to some embodiments, the width of the slot is in the range of 0.5 cm to 2 cm, 0.5 cm to 1.5 cm. or 0.7 cm to 1 cm. Each possibility is a separate embodiment.

According to some embodiments, the slot is tapered, such that a width at the proximal end of the slot is smaller than the width of the slot at its distal end. This may advantageously serve as a guide for the insertion of the IV device. According to some embodiments, the slot may have a stepwise tapered width, each width suitable for a different size of IV device. This may advantageously allow using the patch for differently sized blood vessels, for example.

According to some embodiments, the second layer has an essentially same size and shape as the first layer. According to some embodiments, the second layer includes a second slot having an essentially same size and shape as the slot of the first layer. According to some embodiments, the outer surface of the first layer and/or the inner surface of the second layer may include an adhesive configured to cause the layers to adhere to one another when juxtaposed, after insertion of the IV device.

According to some embodiments, the patch may further include a third layer, the surface of which is configured to be placed over and/or adhered to the first layer and/or the second layer, after adhering the second layer to the first layer.

According to some embodiments, the third layer may be larger (longer and wider) than the first and second layers, so as to fully cover the first and second layers and thus serve as a protective shield.

According to some embodiments, the third layer may include a slit through which part of the insertion device, to which repeated access is required (e.g. the Venflon valve of an infusion needle), may emerge. According to some embodiments, the slit may be narrower (smaller in width) than the slot, thus preventing/reducing infection at the penetration site. Alternatively, the third layer may be integral throughout its surface, i.e. devoid of slits/slots. In such case the third layer is preferably attached to the first and second layer in a reversible and repeatable manner (e.g. Velcro-type attachment), such that it can be lifted, when access to the insertion device is required (e.g. when an infusion tube is provided) and re-adhered upon completion.

According to some embodiments, the first, second and third layers form a single integrative patch.

According to some embodiments, the patch may be used for inserting a needle into blood vessels close to the surface of the skin. According to some embodiments, the patch may be used for inserting a needle of medical devices used in intravenous therapies (e.g. intravenous drips), fluid administration, correcting electrolyte imbalances, delivering medications, and for use in blood transfusions, etc. According to some embodiments, the patch may be used to assist a health care professional in accurately inserting a needle into sensitive areas on the patient's body, such as into a blood vessel in the neck of a patient, particularly in children. According to some embodiments, the patch may be used in a variety of medical military uses. According to some embodiments, the patch may be used for training general medical staff and health care professionals in venipuncture. According to some embodiments, the patch may be used in collecting cerebrospinal fluid (CSF) in which case the slot may serve to mark the insertion point of a needle along the spine for a lumbar puncture, for example.

According to some embodiments, there is provided a method for inserting and fixating an intravenous (IV) device, the method including providing a layered patch including a first layer and a second layer; wherein the first layer includes a slot; removably adhering at least a portion of the first layer to the patient's skin, such that a target blood vessel is exposed in the slot; inserting the IV device into the blood vessel exposed by the slot; and placing the second layer over the first layer, such that a non-inserted portion of the IV device is immobilized between the first and second layers.

According to some embodiments, the slot has a bulged structure on its lateral edges configured to prevent/inhibit lateral movement of the blood vessel. This may advantageously ensure that the blood vessel does not slip sideways when the IV device presses upon it.

According to some embodiments, the method further includes placing a third layer of the patch over the second layer of the patch, such that an area of the patient's skin larger than the area covered by the first and/or second layers is covered. According to some embodiments, the first, second and optionally third layers are attached at their one edge whereas the opposite edge is separable. This may allow placing/positioning the layers using only one hand.

According to some embodiments, the method further includes exposing and/or pulling a part of the insertion device, to which continued access is required, through a slit in the third layer.

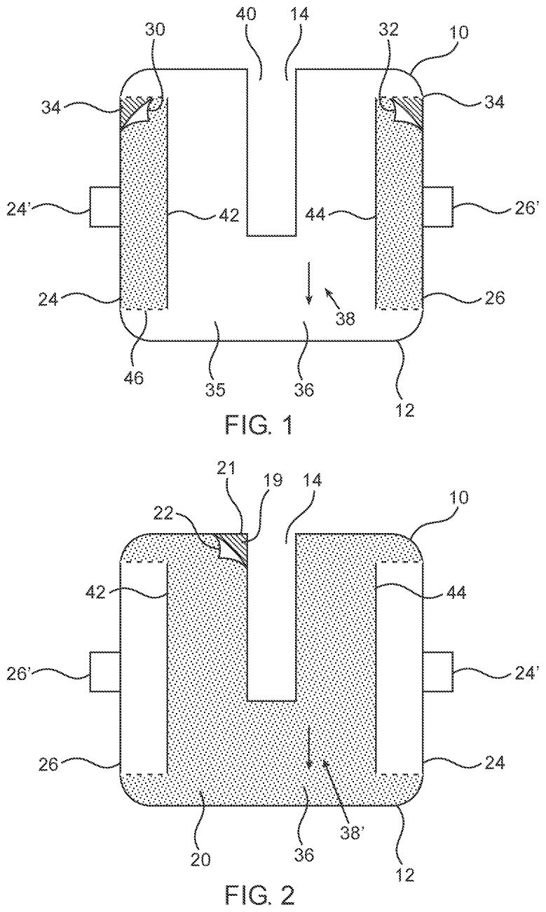

FIG. 1 is a top view of a device 10 used to facilitate insertion of an intravenous (IV) device. Device 10 includes a planar substrate 12, which may be made, for example, from a stiff or rigid material, e.g., plastic, woven fabric or latex rubber, having a slot 14 therein.

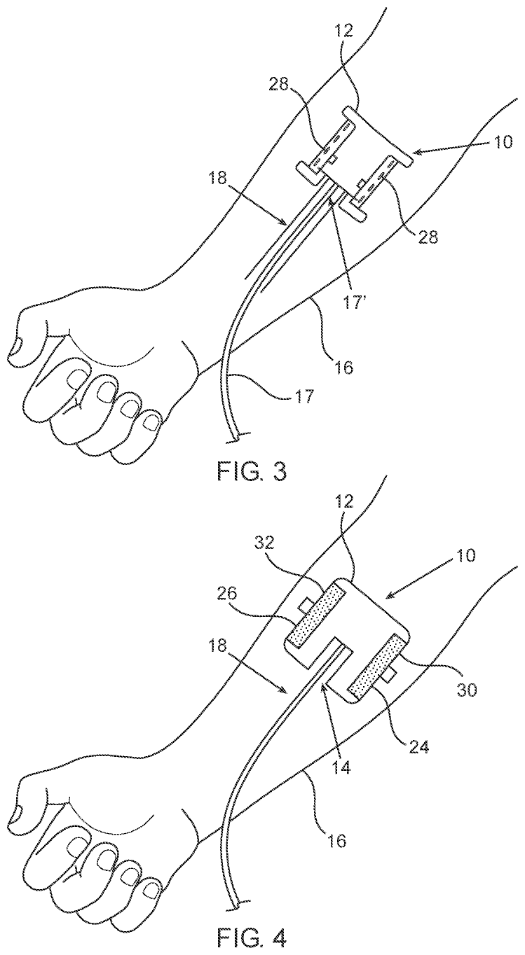

As illustrated in FIG. 3, the substrate 12 may be adhered, for example, to a patient's arm 16, such that a blood vessel 18 is at least partially located within or visible through slot 14. The size and shape of substrate 12 and slot 14 may vary depending on the size of the patient and the size of the blood vessel 18. Substrate 12 improves visualization of the blood vessel 18 and prevents it from sliding sideways when pressure is exerted, for example, by a needle 17′ of an intravenous device 17 inserted into the blood vessel 18. As detailed below, device 12 facilitates insertion and removal of intravenous device 17, such that these steps may be performed one handedly. Device 12 may, for example, may be packaged in sterile form and/or may be sterilizable. The terms intravenous device and medical device may be used interchangeably herein.

As illustrated in the bottom view of the device in FIG. 2, substrate 12 includes an adhesive coating 19 on a first surface 20 of the device 10, which is removably adherable to the patient. A removable film or cover 22 (shown stippled), placed over the adhesive coating 19, maintains the integrity and adhesive quality of the adhesive coating 19 prior to use. A portion of the cover 22 is illustrated lifted at one corner 21 so as to reveal the adhesive layer 19.

The substrate 12 includes flaps 24 and 26. A slot 14 is formed between flaps 24 and 26. Flaps 24 and 26 are connected to each other by a bridge 36. Adhesive coating 19 does not extend onto a portion of first surface 20 extending over flaps 24 and 26. As illustrated in FIG. 1, a top of each of flaps 24 and 26 is coated with an adhesive coating 34. Only the portion of second surface 35 extending over flaps 24 and 26 is coated with adhesive coating 34. Films or covers and 32 (shown stippled), which are placed over adhesive coating 34 on flaps 24 and 26, maintain the integrity and adhesive quality of the adhesive coating 34 prior to use of the flaps 24 and 26. A portion of each of flap covers 30 and 32 is peeled away from flaps 24 and 26 to reveal adhesive coating 34. Flaps 24 and 26 pivot between an initial position where they are coplanar with substrate 12 and a second position illustrated in FIG. 3 where they lie above substrate 12. Flaps 24 and 26 include optional tabs 24′ and 26′ which are easily graspable, thus facilitating pivoting of flaps 24 and 26 to their second position along hinge lines 42 and 44. Dashed lines 46 along both sides of flaps 24 and 26 may represent either a cut or score line. Flaps 24 and 26 may be transparent or translucent, e.g. to allow visualization of the secured intravenous device 17.

As illustrated in FIG. 3, needle 17′ of intravenous device 17 is inserted into the blood vessel 18 through slot 14. The intravenous device 17 may be used both to inject fluids into and to withdraw fluids from the patient and may include a needle, butterfly Intracut, Venflon, etc. Flaps 24 and 26 are folded over the intravenous device 17, for example, over wings 28 of the intravenous device 17, to firmly secure the intravenous device 17 to the patient. Portions of wings 28 indicated in dashed lines are sandwiched between flaps 24 and 26 and remaining portions of substrate 12.

Prior to application of the device 10 to the patient, cover 22 is removed from first surface 20 to expose adhesive coating 19, as illustrated in FIG. 7. The device is then placed on the patient, for example, the patient's arm 16 over blood vessel 18, such that the blood vessel 18 is at least partially visible and accessible through slot 14. After application of the device 10 to the patient's arm, as illustrated in FIG. 4, needle 17′ of intravenous device 17 is inserted into blood vessel 18. The insertion or injection may be conveniently performed using only one hand since the device holds the blood vessel 18 in place, which may conventionally be performed by the user's second hand. After injection or insertion, intravenous device 17 may be fixed in place, if necessary or desired by folding flaps 24 and 26 over wings 28 of the intravenous device 17. Covers 30 and 32 are first removed from flaps 24 and 26, exposing adhesive coating 34 to provide a secure connection of the intravenous device 17 to substrate 12 as illustrated in FIG. 6.

In order to remove intravenous device 17 from the patient, flaps 24 and 26 are lifted or peeled off from wings 28, and intravenous device 17 is pulled out of the blood vessel 18, after which, substrate 12 may be pulled or peeled off the patient. Device 10 may be designed to allow for each insertion and removal step to be performed one handedly.

Substrate 12 may be arranged in any direction on the patient so long as the slot 14 is placed over blood vessel 18. For example, open side 40 of slot 14 may be further from the patient's heart than bridge 36 (open side 40 is located distally to bridge 36). It is noted that the intravenous device 17 is inserted in the direction of the arrows included in indicia 38 on second surface 35 and indicia 38′ on first surface 20 and on cover 22.

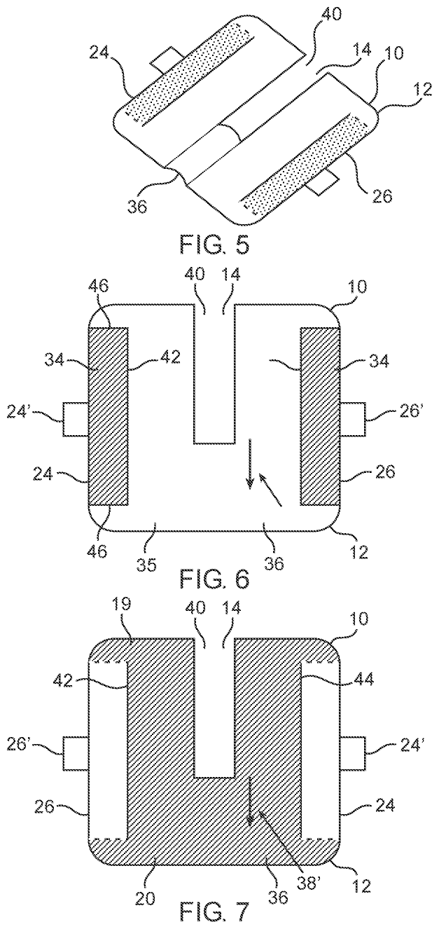

In an exemplary embodiment illustrated in FIG. 5, bridge 36 may include an arch, which is illustrated having a hemispherical shape but may have any other shape as well, including v-shaped, u-shaped, etc.

In an exemplary embodiment, second surface 35 may have an adhesive coating on a central portion of the device between flaps 24 and 26, which directly adheres to an underside of wings 28. In this embodiment, flaps 24 and 26 are optional, given that other portions of substrate 12 are used to secure wings 28 to the device 10.

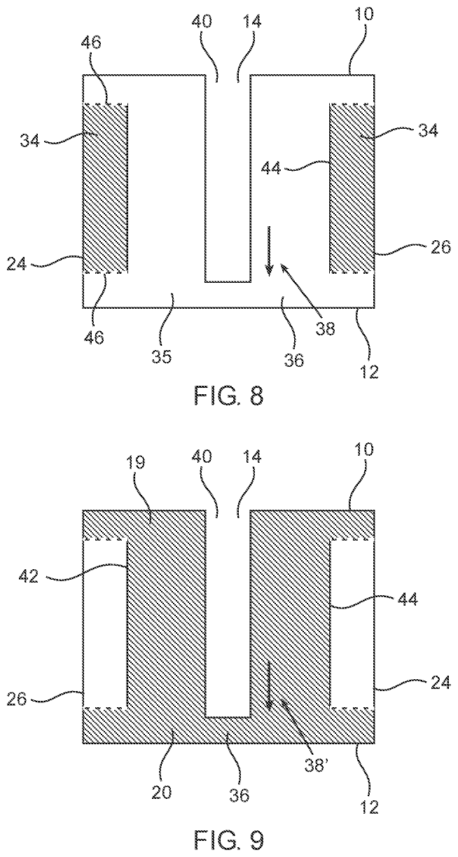

FIG. 8 is top view of an exemplary embodiment of the device 10, which is identical to the embodiment shown in FIG. 1 except that flaps 24 and 26 do not include tabs 24′ and 26′, and that covers 30 and 32 have been removed, exposing adhesive coating 34 on second surface 35 over flaps 24 and 26. Further, the corners of substrate 12 are more angled than in the embodiment illustrated FIG. 1.

FIG. 9 is a bottom view of the device shown in FIG. 6. Cover 22 has been removed exposing adhesive coating 19 on first surface 20.

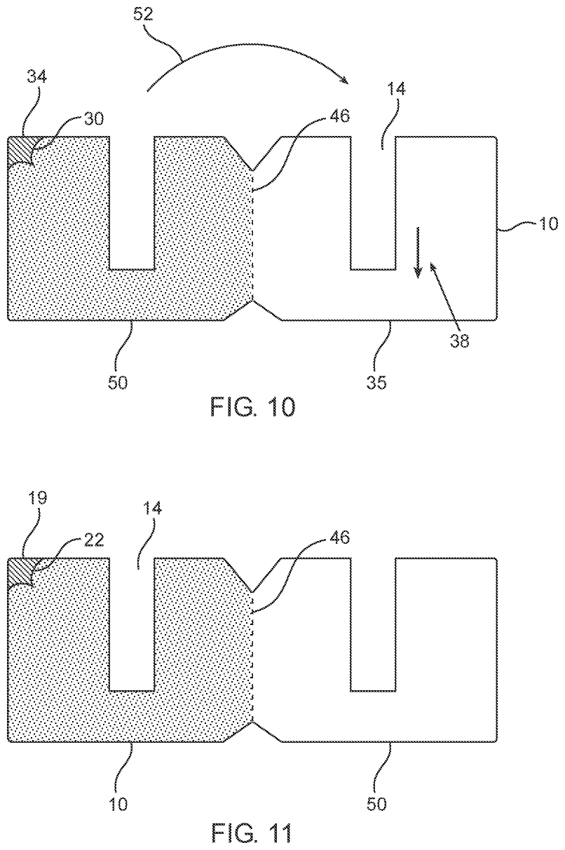

FIG. 10 is a top view of an exemplary embodiment of the device 10. A single larger flap 50, which may be transparent or translucent, e.g., to permit visualization of the intravenous device 17, is used instead of the two smaller flaps 24 and 26 illustrated in FIGS. 1 to 8. A width of the flap may correspond substantially to a width of the portion of device 10 adhered to the patient. Alternatively, the width of the flap 50 may be at least sufficient to lie over the slot 14 and the intravenous device 17 after being folded over, as described below. After cover 22 is removed from a bottom surface of the device 10, shown in FIG. 11, adhesive 19 is used to removably adhere the device 10 to the patient. Next, the intravenous device 17 is inserted into the patient through slot 14, and cover 30 is removed, exposing adhesive 34. Flap 50 is then folded in the direction of arrow 52 (e.g., to the right as illustrated in FIG. 10 along score line 46 over surface 35 and an exposed portion of the intravenous device 17 lying over surface 35. As an alternative to or addition to the use of adhesive 34 on flap 50, surface 35 may have an adhesive coating for securing an underside of the intravenous device.

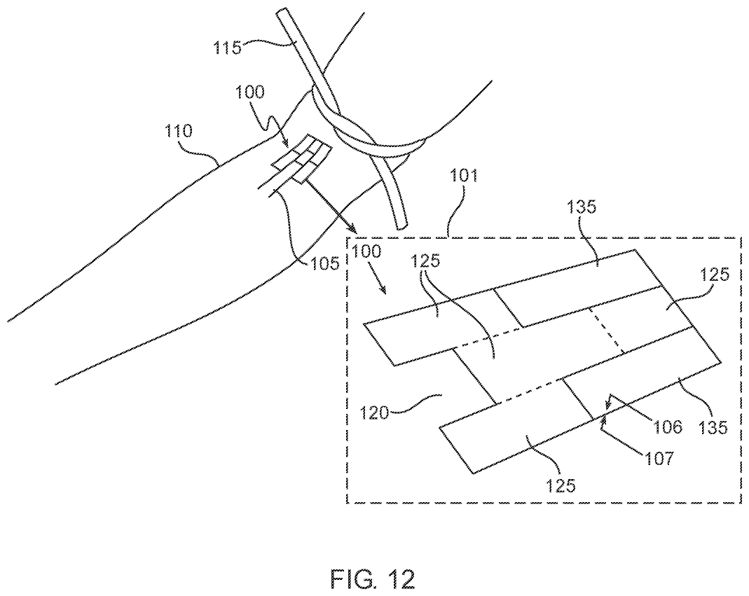

FIG. 12 schematically illustrates a top view of a device 100 for facilitating insertion of a medical device into a blood vessel 105 of a patient, according to an exemplary embodiment of the present invention. A slot 120 in a substrate 135 of device 100 may be placed over blood vessel 105 of the patient. A tourniquet 115 is placed around the arm, typically at the bicep of the patient to facilitate finding a viable vein to use. Blood vessel 105 will be more visible within slot 120 when a tourniquet, such as tourniquet 115, is used.

An expanded view 101 of device 100 in FIG. 12 illustrates a medical device flap 125 positioned flush against substrate 135. The dotted region of medical device flap 125 represents a portion of medical device flap 125 above slot 120. A bottom side 107 of device 100 may be coated with an adhesive for removably attaching substrate 135 of device 100 to arm 110 of the patient medical device flap 125 above slot 120.

Slot 120 of device 100 may be used to prevent the lateral sliding of blood vessel 105 such as a vein by anchoring blood vessel 105 when the health care professional, such as a doctor, nurse and/or lab assistant, for example, attempts to insert the needle of medical device 100 into a lumen of blood vessel. Without slot 120 anchoring blood vessel 105, the blood vessel may move and the needle may miss entering the lumen of the blood vessel during the attempted venipuncture. Moreover, slot 105 of device 100 may assist the health care professional in marking the position of the target blood vessel for venipuncture.

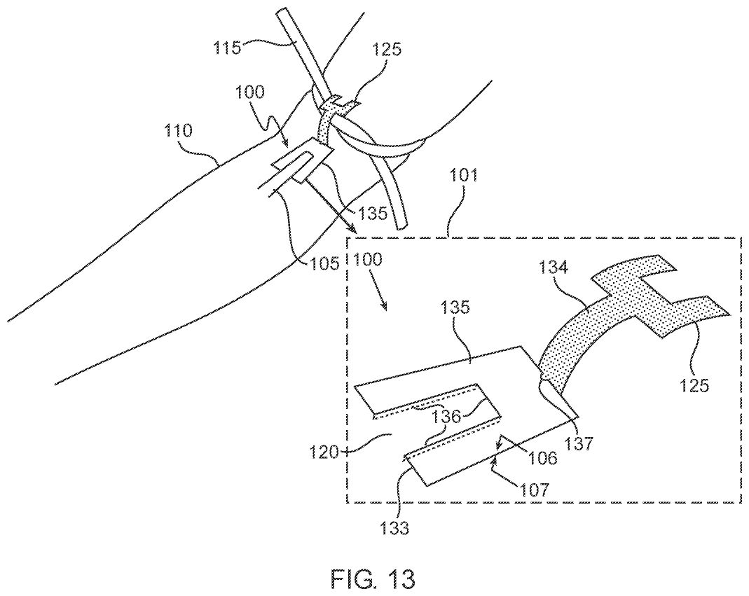

FIG. 13 schematically illustrates a device for facilitating insertion of a medical device into a blood vessel of a patient with medical device flap 125 pulled upward, according an exemplary embodiment of the present invention. Medical device flap 125 may include a removable flap cover 134 at least partially covering an adhesive on medical device flap 125. Removable flap cover 134 may include a pull tab 137 for easily removing flap cover 134 from medical device flap 125. When medical device flap 125 is pulled upward above substrate 135, slot 120 may be entirely exposed so as to help the health care professional locate and mark blood vessel 105. In some embodiments, walls 136 forming slot 120 in substrate 135 may be configured to be rigid so as to firmly hold blood vessel 105 while inserting the needle into the blood vessel during the attempted venipuncture.

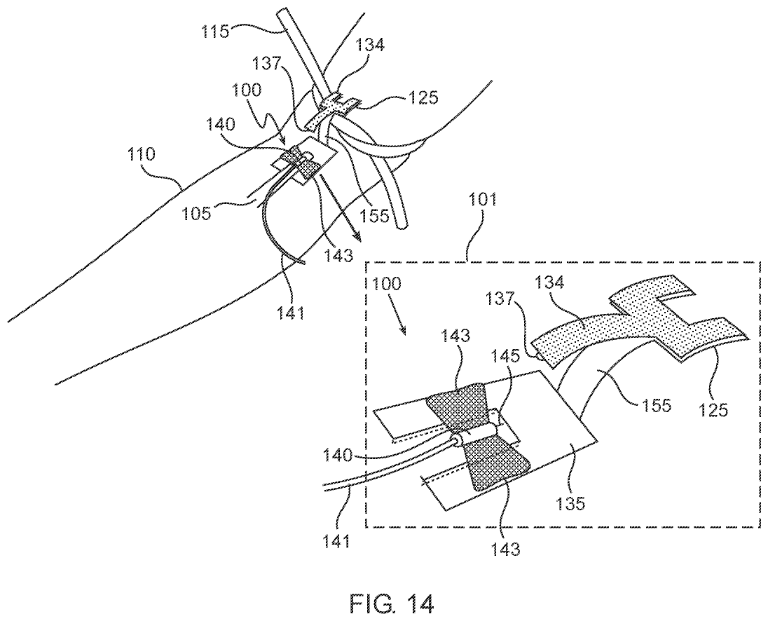

FIG. 14 schematically illustrates a device for facilitating insertion of a medical device into blood vessel 105 of a patient after insertion, according to an exemplary embodiment of the present invention. Medical device 140 may include a needle 145, butterfly wings 143, and a tube 141. Needle 145 may be configured to be inserted into blood vessel 105 during venipuncture, as shown in the enlarged view of FIG. 14. Tube 141 may be used, for example, to collect blood drawn from blood vessel 105 in the embodiment shown in FIG. 14. Pull tab 137 may be pulled for removing flap cover 134, thereby exposing a surface 155 at least partially coated with an adhesive on medical device flap 125.

In some embodiments, medical device flap 125 may be Y-shaped.

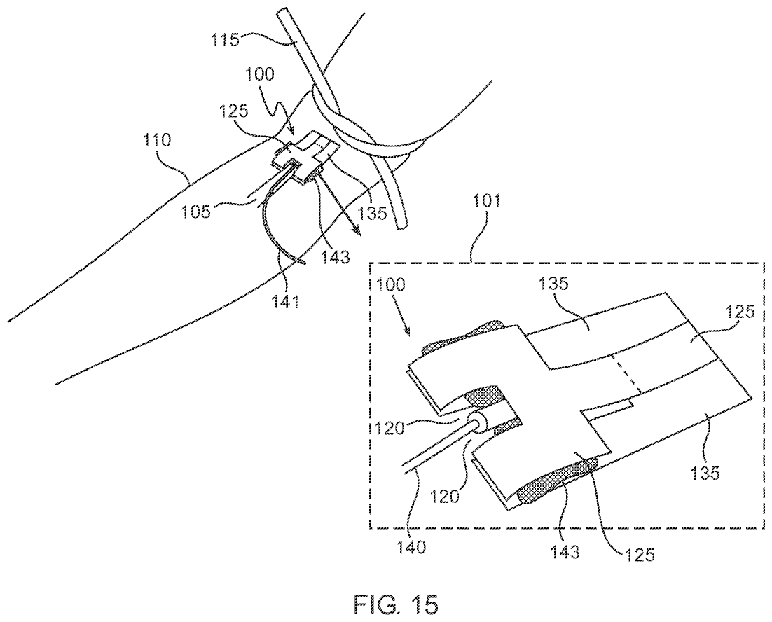

FIG. 15 schematically illustrates a device for facilitating insertion of a medical device into blood vessel 105 of a patient after insertion according to an exemplary embodiment of the present invention. FIG. 15 illustrates a medical device flap 125 folded downward and adhering to a medical device 140 so as to fix medical device 140 and needle 145 in place. In some embodiments, device 100 for facilitating insertion of a medical device 140 into blood vessel 105 may be placed in any suitable position on the patient's body and is not limited to being placed on arm as shown in FIGS. 12-15. Slot 120 of device 100 may be configured to mark an area on arm for the health care professional to insert needle 145. Rigid walls 136 of slot 120 may, for example, be used to fix blood vessel 105 in place during venipuncture and to maintain the position of blood vessel 105 from possible movements of blood vessel due to changes in blood pressure.

In various embodiments, device 100 may be used mainly for inserting needle 145 into blood vessels close to the surface of the skin. Device may be used to insert needle 145 of medical devices 140 used in intravenous therapies (e.g. intravenous drips) such as for fluid administration (when treating dehydration, for example), to correct electrolyte imbalances, to deliver medications, and for use in blood transfusions, for example. Device 100 may be used to assist health care professionals in accurately inserting needle 145 into sensitive area in the patient's body, such as a blood vessel in the neck of the patient, particularly in children. Device 100 may be used in a variety of medical military uses. Device 100 may be used for training general medical staff and health care professionals in venipuncture. Device 100 may be used in collecting cerebrospinal fluid (CSF) where using slot 120 to mark the insertion point of needle 145 along the spine for a lumbar puncture, for example, may be critical.

In some embodiments, device 100 may be a patch and/or a sticker. Any suitable geometries (e.g., patch length, patch width, slot length and/or slot width) of device 100 may be chosen, for example, to account for the age of the patient and/or for different use cases.

In some embodiments, substrate 135 may include an edge such that slot 120 is formed from three walls 136 of substrate 135 as shown in FIG. 13. In other embodiments (not shown in the figures), slot 120 may be formed in the interior of the patch such that slot 120 may include four walls 136 of substrate 135. In other embodiments (not shown in the figures), slot 120 may be formed in a V shape such that slot 120 may include two walls 136 of substrate 135.

According to some embodiments, walls 136 may extend from slot 120 perpendicularly to substrate 135. According to some embodiments, the slot may be three-dimensional having any shape formed through device 100 from a front side 106 to bottom side 107, such as through substrate 135, for example, as shown in FIG. 12 and FIG. 13.

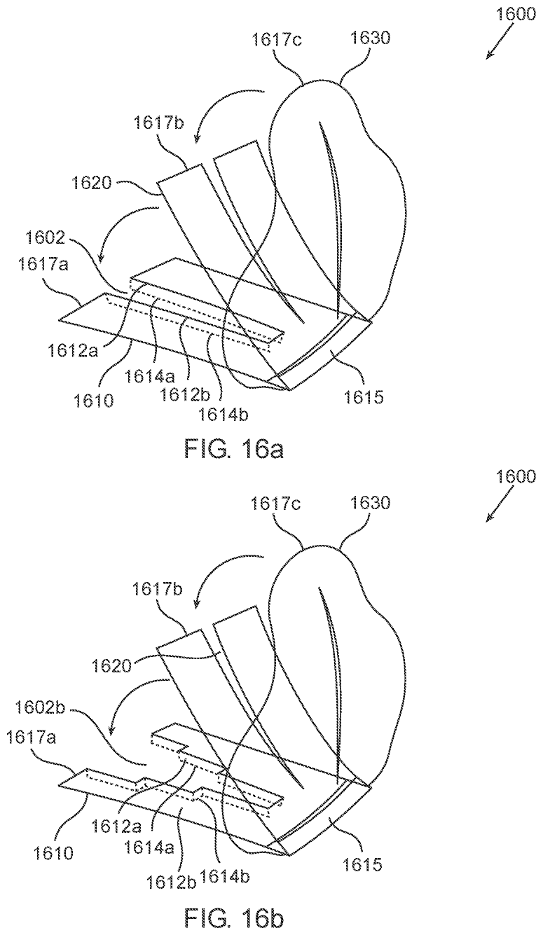

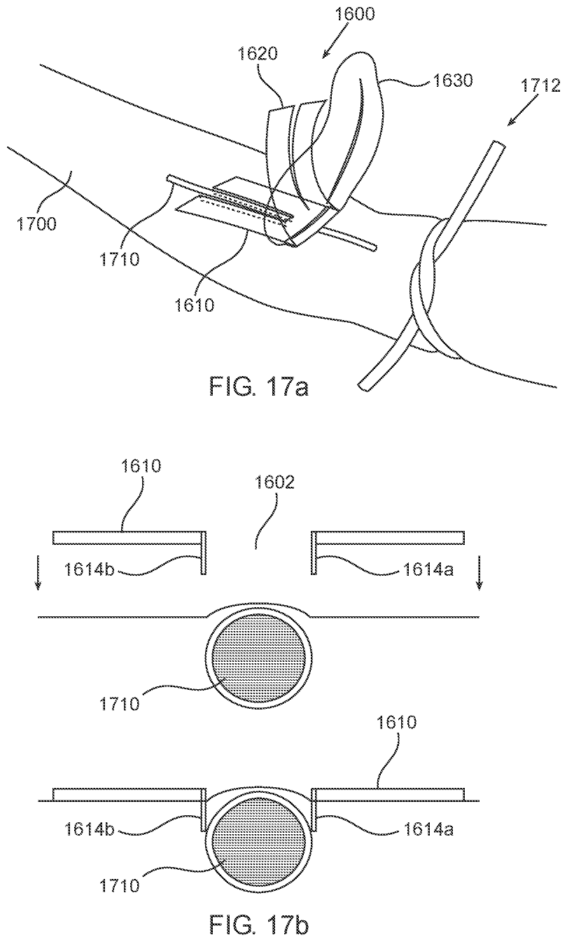

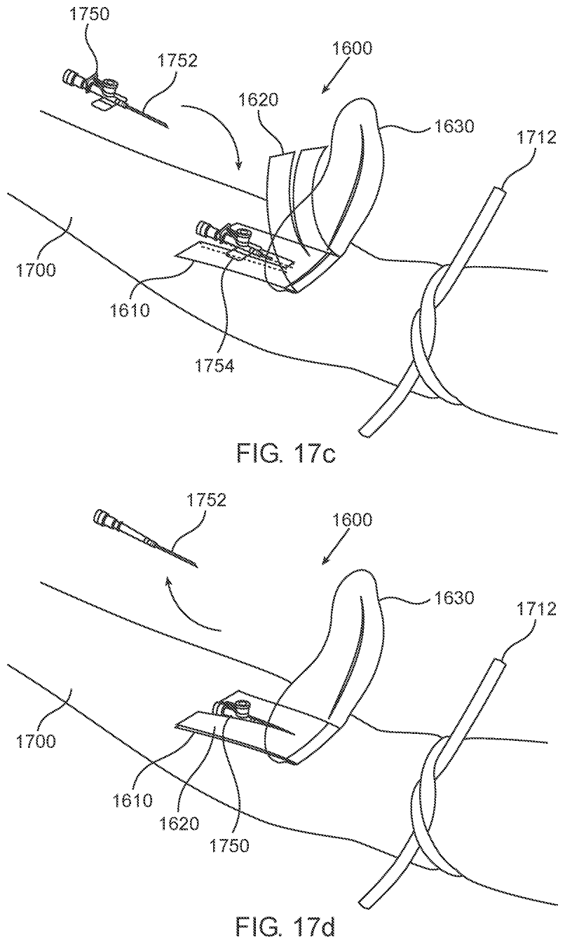

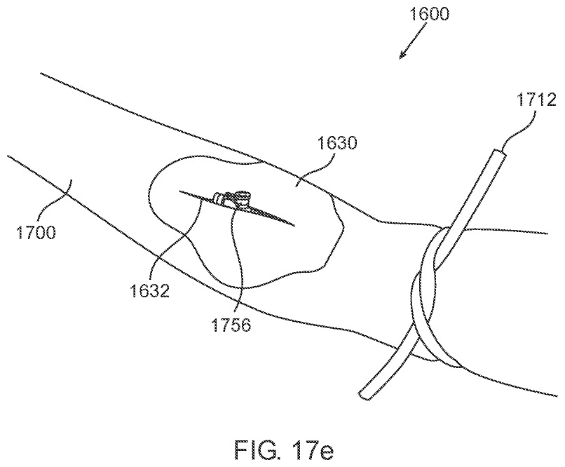

Reference is now made to FIG. 16a and FIG. 16b which schematically illustrate a layered patch 1600 for inserting and fixating an intravenous (IV) device 1750, according to an exemplary embodiment of the present invention, and to FIGS. 17a-17d, which schematically shows layered patch 1600 at different stages of inserting IV device 1750.

Patch 1600 includes a first layer 1610 configured to adhere to a patient's skin, and a second layer 1620 configured to be placed over and/or adhere to first layer 1600 and a third layer 1630. First layer 1610, second layer 1620 and third layer 1630 are attached one to another at a first end 1615 of patch 1600, whereas opposite ends 1617a, 1617b and 1617c are separate (non-attached). The first layer 1610 includes a slot 1602 sized and shaped to expose a blood vessel 1710 (shown in FIG. 17a) and to allow essentially unhindered access of the IV device 1750 to the exposed blood vessel 1710, when adhered to the patient's skin (as best seen in FIG. 17c). The lateral edges 1612a and 1612b of slot 1602 include flaps 1614a and 1614b (also referred to herein as bulked structure) configured to protrude from the lower surface of first layer 1610. According to some embodiments, slot 1602 may include a stepwise tapering (referred to as 1602b in FIG. 16b) configured to allow capturing blood vessels of different sizes, thus enabling secure insertion of IV device 1650 for differently sized vessels such as vessels of small children and infants.

Patch 1600 is preferably packaged in sterile form. Additionally or alternatively, patch 1600 may be sterilizable. According to some embodiments, patch 1602 may include an adhesive coating/cover (not shown) configured to cover the adhesive surfaces of patch 1600.

As illustrated in FIG. 17a, first layer 1610 may be adhered to a subject's skin for example on a patient's arm 1700, such that a blood vessel 1710 is at least partially located in slot 1602 between flaps 1614a and 1614b. As best seen in FIG. 17b, flaps 1614a and 1614b are configured to form a tunnel-like structure adapted to prevent/inhibit lateral movement of blood vessel 1710, as best seen in FIG. 17b. This is of particular advantage when a tourniquet 1712 is applied and the vein becomes rigid, with a tendency to ‘jump’ sideways when pressure is applied thereon (e.g., when touched by a needle).

The size and shape of patch 1600 and slot 1602 may vary depending on the size of the patient and the size of the target blood vessel. As detailed below, device 1600 facilitates insertion and removal of intravenous device 1750, such that these steps may be performed one handedly.

As illustrated in FIG. 17c, needle 1752 of IV device 1750 is inserted into blood vessel 1710 through slot 1602. Intravenous device 1750 may be used to inject fluids into and/or to withdraw fluids from the patient and may, as here, include a needle, butterfly, Intracut, Venflon, etc. Upon insertion of IV device 1750, needle 1752 may be withdrawn and second layer 1620 may, as shown in FIG. 17d, be laid over and adhered to first layer 1610 such that wings 1754 of IV device 1750 are sandwiched between first layer 1610 and second layer 1620, thereby firmly securing intravenous device 1750 to the patient.

Finally, as shown in FIG. 17e, third layer 1630 may be laid over and adhered to second layer 1620 and to a portion of the subject's skin surrounding first layer 1610 and second layer 1620, so as to protect the area of insertion from contaminations etc. Third layer 1630 includes a slit 1632, through which inlet port 1756, to which repeated access is required, can emerge. Optionally, at least the outer surface of third layer 1630 may be made from a water-proof material, so as to provide optimal protection.

According to some embodiments, the terms “stabilize”, immobilize” and “anchor” may be used interchangeably.

According to some embodiments, the terms “intravascular”, intra-blood vessel” and “IV” may be used interchangeably.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description and is not intended to be exhaustive or to limit the invention to the precise form disclosed. It should be appreciated by persons skilled in the art that many modifications, variations. substitutions, changes, and equivalents are possible in light of the above teaching. It is, therefore to be understood that the appended claims are intended to cover all such modifications and changes which fall within the true spirit of the invention.

While certain features of the invention have been illustrated and described herein, many modifications, substitutions, changes and equivalents will now occur to those of ordinary skill in the art. It is therefore, to be understood that the appended claims are intended to cover all such modifications and changes as fall within the true spirit of the invention.

1.-27. (canceled)

28. A layered patch for inserting and fixating an intravenous (IV) device, the patch comprising:
a first layer configured to adhere to a patient's skin, said first layer comprising a slot configured to expose a blood vessel of the patient, wherein said slot is sized and shaped to allow essentially unhindered access of the IV device to the exposed blood vessel and wherein lateral edges of said slot comprise a bulged structure configured to prevent/inhibit lateral movement of the exposed blood vessel; and
a second layer configured to be placed over and/or adhere to said first layer, wherein said first and second layers are attached one to another at a first end thereof.

29. The patch of claim 28, wherein said first layer is coated with an adhesive configured to removably adhere to the patient's skin on a first, patient facing surface thereof.

30. The patch of claim 28, wherein a width of the slot is greater than a width of the blood vessel.

31. The patch of claim 30, wherein the width of the slot is in the range of 0.5 cm to 1.5 cm.

32. The patch of claim 28, wherein said slot is close at its proximal end and open at its distal end.

33. The patch of claim 32, wherein said slot is tapered, such that a width at the proximal end of the slot is smaller than the width of the slot at its distal end.

34. The patch of claim 33, wherein said tapering is stepwise.

35. The patch of claim 28, wherein said second layer has a same size and shape as said first layer.

36. The patch of claim 28, wherein said second layer comprises a second slot having essentially a same size and shape as the slot of the first layer.

37. The patch of claim 28, further comprising a third layer configured to be placed over and/or adhered to said first layer and/or said second layer.

38. The patch of claim 37, wherein said third layer is larger than the first and second layers.

39. The patch of claim 38, wherein said third layer comprises a slit through which part of the insertion device, to which repeated access is required, may emerge.

40. The patch of claim 39, wherein said slit is narrower than said slot, thus preventing/reducing infection at the penetration site.

41. The patch of claim 37, wherein said third layer is attached to the first and/or second layers at the first end thereof.

42. The patch of claim 28, wherein the first, second and third layers form a single integrative patch.

43. The patch of claim 28, wherein the first, second and/or third layers comprise a luminescent material facilitating the insertion and/or fixation of the intravenous (IV) device.

44. A method for inserting and fixating an intravenous (IV) device, the method comprising:
providing a layered patch comprising a first layer and a second layer; wherein said first layer comprises a slot;
removably adhering at least a portion of the first layer to the patient's skin, such that a target blood vessel is exposed in the slot;
inserting the IV device into the blood vessel exposed by the slot; and
securing the IV device to the patient's skin by placing the second layer over the first layer, such that a non-inserted portion of the IV device is immobilized between the first and second layers.

45. The method of claim 44, wherein the slot comprises a bulged structure on its lateral edges configured to prevent/inhibit lateral movement of the blood vessel.

46. The method of claim 44, further comprising: placing a third layer of the patch over the second layer of the patch, such that an area of the patient's skin larger than the area covered by the first and/or second layers is covered; and exposing and/or pulling a part of the insertion device, to which continued access is required, through a slit in the third layer.

47. A method for producing a layered patch for inserting and fixating an intravenous (IV) device, the method comprising:
forming a first layer comprising a slot sized and shaped to expose a blood vessel of a patient and to allow essentially unhindered access of the IV device to the exposed blood vessel, when adhered to a patient's skin; wherein lateral edges of the slot comprise a bulged structure configured to prevent/inhibit lateral movement of the exposed blood vessel;
forming a second layer configured to be placed over and/or adhere to said first layer, attaching edges of the first and second layers at a first end thereof.
